(19) United States
(12) Patent Application Publication
(10) Pub. No.: US 2017/0216461 A1
(43) Pub. Date: Aug. 3, 2017

(54) Composite Nanodots Based on Carbon Nanodots and Preparation Method Thereof

(71) Applicant: Wenzhou Institute of Biomaterials and Engineering, Wenzhou (CN)

(72) Inventors: Zhe Liu, Wenzhou (CN); Qien Xu, Wenzhou (CN); Yuanhui Song, Wenzhou (CN); Yihong Li, Wenzhou (CN)

(21) Appl. No.: 15/011,685

(22) Filed: Feb. 1, 2016

(51) Int. Cl. A61K 49/00

(52) U.S. Cl. CPC A61K 49/0093; A61K 49/003

(57) ABSTRACT

The present invention discloses a preparation technique of composite nanodots based on carbon nanodots, and their use in the field of fluorescent imaging, wherein, the main components of the composition are carbon nanodots, which are material with superior biocompatibility characteristics, and supporting component is methylene blue, and particle diameter range is 100-500 nanometers, and the zeta potential is −35 to 10 millivolts. The above said techniques for preparation of composite nanodots are safe, quick and simple, low cost, and easy to perform for industrialized production. Composite carbon nanodots have good biocompatibility and safety, high fluorescence imaging sensitivity, and they are promising in gaining wider use in the fields of biomedical imaging, targeting diagnosis and therapy, drug screening and optimization, and in vivo labelling and tracing, and have potential value in personalized medicine.

### BACKGROUND OF THE INVENTION

(a) Field of the Invention

The present invention relates composite carbon nanodots used in fluorescent biological imaging technique and their preparation and purification methods.

(b) Description of the Related Art

Fluorescent imaging is an in vivo and in vitro biomedical optics imaging technique newly developed in the last decade. Since this technique is simple and convenient to use, has low harm on living organisms, provides the advantages of direct imaging result, rapid measurement, high sensitivity, and low measurement cost, it became an ideal method for in vitro imaging of living organism and in vivo imaging of small animals. Using this kind of imaging technique, it is possible to directly and instantaneously observe the in vivo distribution of marked genes or cells in the bodies of animals or involved organisms, pathological processes and responses. Therefore, it is widely used in the researches on development and progression of various kinds of diseases, mode of actions of drugs and their metabolism in the body, and screening of new drugs. Fluorescent probe is the core component of fluorescent imaging technique, as it directly resolves the imaging position, imaging time, and image resolution after reconstruction. As a result, the fluorescent imaging technique mainly depends on the continuous development and functionalization of fluorescent probes. Currently, the most common fluorescent probes are organic fluorescent dyes and quantum dots. However, organic fluorescent dyes have drawbacks such as low resistance to photobleaching and short half lives. On the other hand, although quantum dots have intrinsically better qualities in terms of these two aspects, they have low biocompatibility and high cytotoxicity, and therefore, their further extensive use is limited.

Carbon nanodots are attracting more and more attention due to their benefits such as chemical inertness, lack of optical scintillation, low photobleaching rates, low toxicity, and good biocompatibility. Carbon nanodots can be used in various fields such as biological imaging, photocatalysis, detection, lasers, LEDs, power storage, and transformation devices. Recent developments in the field of carbon nanodot synthesis methods would enable top-down production method via improved carbon structures (such as graphene, multi-walled carbon nanotubes) or down-top production method via chemical substances comprising carbon (such as ammonium citrate and EDTA). It is noteworthy that, the carbon nanodots obtained with these methods all require surface oxidation or purification, so that they can be luminous and water-soluble. Besides, carbon nanodots with surface purification obtained with a single-step method (such as microwave reaction method) have also been reported. These methods have the advantages of quick initiation, easy control of heating, and homogeneous heating process.

In recent years, near infrared fluorescent probes have become a focus of studies, since they have big signal-to-noise ratio, strong background interference, and strong biological penetration characteristics (can perform imaging on deep tissues). Methylene Blue, used in the present invention is a US Food and Drug Administration-approved (FDA-approved) fluorescent imaging dye. Its emission wavelength is around 700 nanometers, and therefore it belongs to the near infrared region. However, since these kinds of dyes have the drawbacks of relatively low stability in the body and short metabolism cycles, they require a carrier to load the dye molecules for performing final clinical applications. The combination of the carbon nanodots and the fluorescent dyes form composite fluorescent carbon nanodots, which are useful in ultimate realization of these composite biomaterials in extensive applications in the fields of biological imaging and clinical imaging.

### SUMMARY OF THE INVENTION

The primary purpose of the present invention is to provide composite nanodots based on carbon nanodots in order to overcome the shortcomings and deficiencies of the prior art. The composite nano-fluorescence imaging materials have good biocompatibility and safety, high fluorescence imaging sensitivity, and they are promising in gaining wider use in the fields of biomedical imaging, targeting diagnosis and therapy, drug screening and optimization, and in vivo labelling and tracing, and have potential value in personalized medicine.

The secondary purpose of the present invention is to provide a safe, quick, simple, and low cost single-step method for preparing water-soluble dye carbon nanodots via microwave radiation and a highly effective separation and purification method thereof, and thus load the obtained carbon nanodots on methylene blue molecules in order to ensure high-sensitivity fluorescence imaging. The above said techniques for preparation and purification of composite nanodots are safe, quick and simple, low cost, and easy to perform industrialized production.

In order to achieve the first purpose of the present invention, the present invention provides a nano-composite material with the main component of carbon nanodots and with fluorescent dye molecules loaded on these components. Moreover, the carbon sources used in preparation of carbon nanodots can be any of wolfberry leaching agents, soy milk, and dietary milk etc., the loading component is methylene blue, and the grain diameter range is 100-500 nanometers, the zeta potential range is −30 to 10 millivolts, and the concentration of methylene blue in the composite nanodots is 1-10 micrograms/milliliter.

In order to achieve the second purpose of the invention, the present invention provides the following technical proposal: A preparation method of composite nanodots based on carbon nanodots, characterized in that; it comprises the operation steps of:
a. forming a solution of the above said carbon sources and 0.5 milligrams/millilitres of methylene blue by means of mixing in the ratio of 0.1:1 to 10:1 by volume, diluting the mixture 1-10 folds with ultra-pure water, and thus obtaining a precursor solution;
b. placing the above said precursor solution in a microwave reaction instrument, and setting the instrument parameters as follows: temperature: 100-180° C., time: 30-300 minutes;
c. reacting for more than 30 minutes, obtaining a clear solution as a result of centrifugal separation, and obtaining crude product of composite carbon nanodots;
d. applying purification on the crude product of composite nanodots. Purification can be applied on the above said crude product of composite nanodots using ultrafiltration or dialysis method, and thus composite nanodots based on carbon nanodots that can be used in fluorescent imaging can be obtained.

The method of water soluble carbon nanodot preparation via microwave irradiation according to the present invention is completely performed in aqueous solution, is safe, easy, quick and simple to apply, has low toxicity, and its raw materials are easy to obtain. The water-soluble carbon nanodots obtained after dialysis or ultrafiltration purification have beneficial monodispersion, good fluorescence and quantum yield, good stability, good water solubility characteristics, and can be widely used as fluorescent marker in biological detection and analysis applications. The development of medical fluorescence imaging materials and expanding the preparation technique of fluorescent contrast agents are of great importance in the field of biomedical fluorescent imaging.

The figures attached to the specification and the below given specific embodiment are given for the purpose of describing the invention better.

### BRIEF DESCRIPTION OF THE DRAWINGS

FIG. 1 is a flow chart of the preparation technique of composite carbon nanodots;

FIG. 2a shows the particle size distribution of the composite carbon nanodots given in embodiment 1;

FIG. 2b shows the zeta potential phenogram of the composite carbon nanodots given in embodiment 1;

FIG. 3a shows the particle size distribution of the composite carbon nanodots given in embodiment 2;

FIG. 3b shows the zeta potential phenogram of the composite carbon nanodots given in embodiment 2;

FIG. 4a shows the particle size distribution of the composite carbon nanodots given in embodiment 3;

FIG. 4b shows the zeta potential phenogram of the composite carbon nanodots given in embodiment 3;

FIG. 5a shows the fluorescence spectogram of the composite carbon nanodots given in embodiment 1 at the excitation wavelength between 340-440;

FIG. 5b shows the fluorescence spectogram of the composite carbon nanodots given in embodiment 1 at the excitation wavelength of 650;

FIG. 6a shows the fluorescence spectogram of the composite carbon nanodots given in embodiment 2 at the excitation wavelength between 340-440;

FIG. 6b shows the fluorescence spectogram of the composite carbon nanodots given in embodiment 2 at the excitation wavelength of 650;

FIG. 7a shows the fluorescence spectogram of the composite carbon nanodots given in embodiment 3 at the excitation wavelength between 340-440;

FIG. 7b shows the fluorescence spectogram of the composite carbon nanodots given in embodiment 3 at the excitation wavelength of 650;

FIG. 8 is a comparison chart of the in vitro fluorescent imaging effects of only the carbon nanodots and the composite carbon nanodots;

FIG. 9 is a comparison chart of fluorescent imaging effects of intravenous injection of the composite carbon nanodots of embodiment 1 on rat tail, before injection and 3.5 hours after injection.

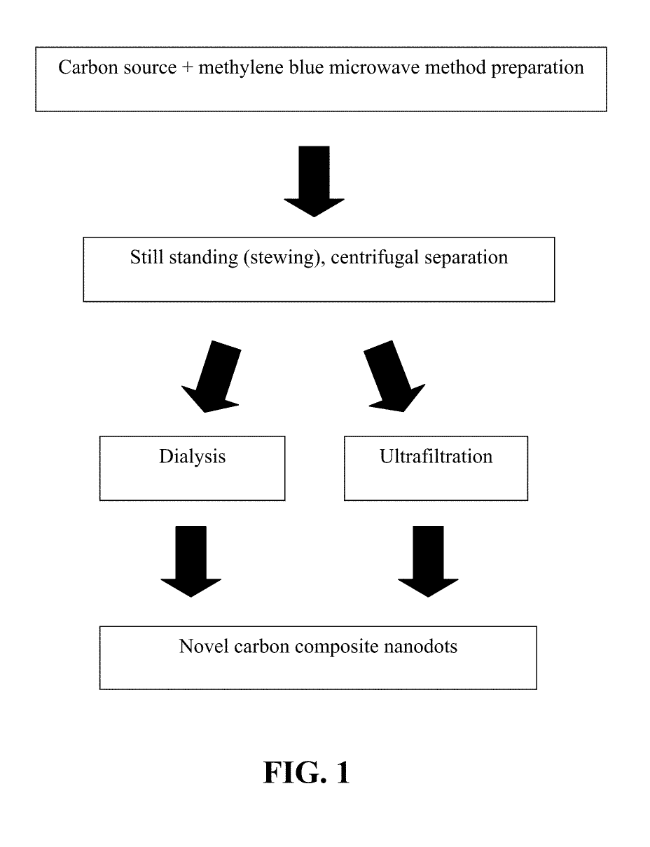

### DETAILED DESCRIPTION OF THE PREFERRED EMBODIMENTS

Below given specific descriptions about the present invention through embodiments are only for better understanding of the invention, and do not form any limitation in the protection scope of the invention disclosed in the claims, and persons skilled in the related technical field can make some non-essential changes and modifications on the present invention according to the contents of the above said description.

The raw materials used in the preparation of the present invention are all commercially available.

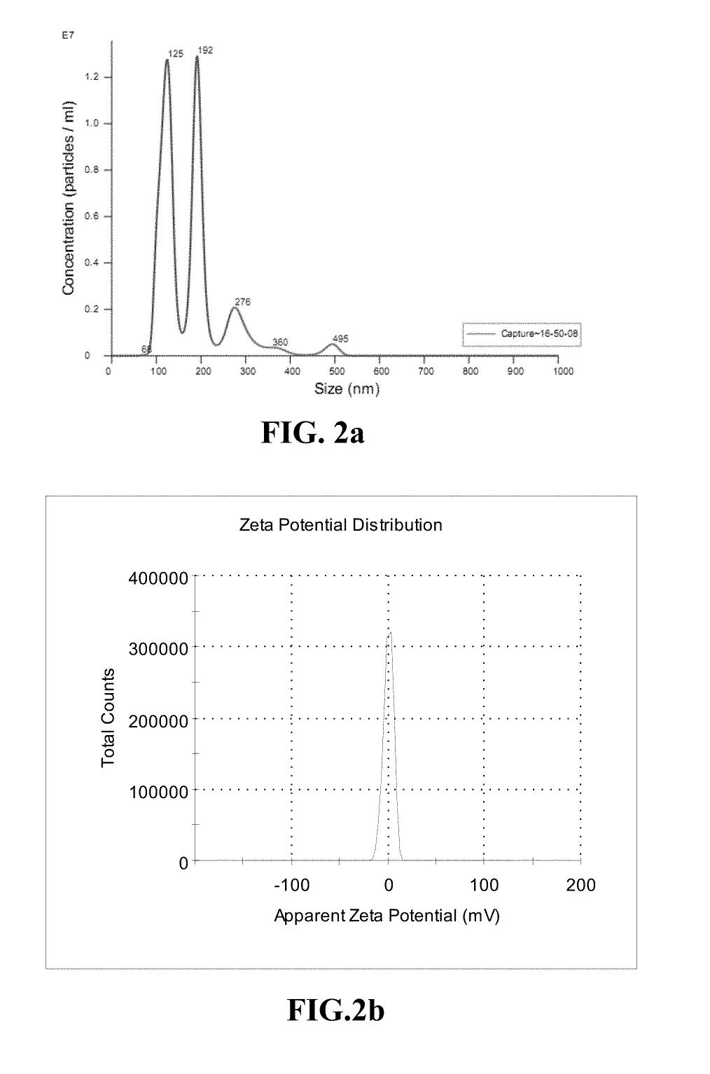

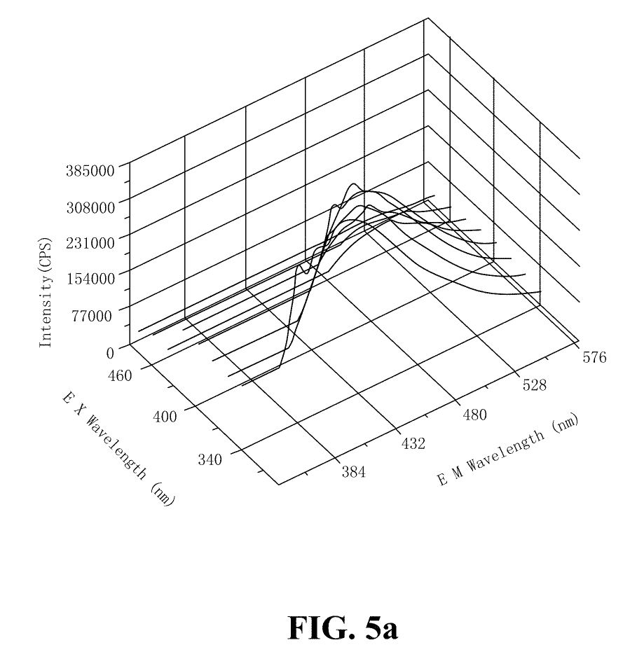

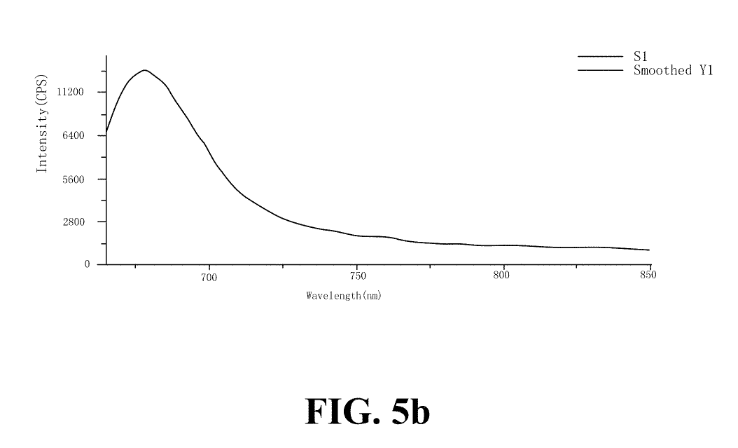

Embodiment 1: 2 milliliters of wolfberry leaching agent and 2 milliliters of methylene blue solution (0.5 milligrams/milliliters) are mixed, and then diluted 1 fold using ultra-pure water to obtain a precursor solution. The precursor solution is then placed in a 5 milliliter special glass bottle for use in microwave reaction instruments, and afterwards, the bottle is placed in a microwave reaction instrument, and the reaction conditions are set to 180 degrees centigrade and 30 minutes. The reaction system is allowed to wait for 50 minutes, and then centrifugated, and the supernatant liquid is retained, and composite carbon nanodots are obtained following ultrafiltration. The methylene blue concentration measured in composite nanodots using ultraviolet spectrophotometer standard curve method is 5.6 micrograms/milliliter. Measurements are made by a laser particle analyzer according to dynamic light scattering principle, and the composite carbon nanodot grain size distribution is found as 179±77.1 nanometers (See, FIG. 2a), while the surface zeta potential is found as 6.85±2.20 millivolts (See, FIG. 2b). The fluorescence properties of composite carbon nanodots are measured by a fluorescence detector device Imaging of composite carbon nanodots and unloaded blank comparative groups in a fluorescence field confirmed that the composite carbon nanodots can remarkably increase the contrast and resolution of fluorescent imaging Under extremely low concentrations, relatively strong fluorescent response signal is obtained (See FIGS. 5a and 5b), which can be used in the fields of bio-labelling and medical imaging.

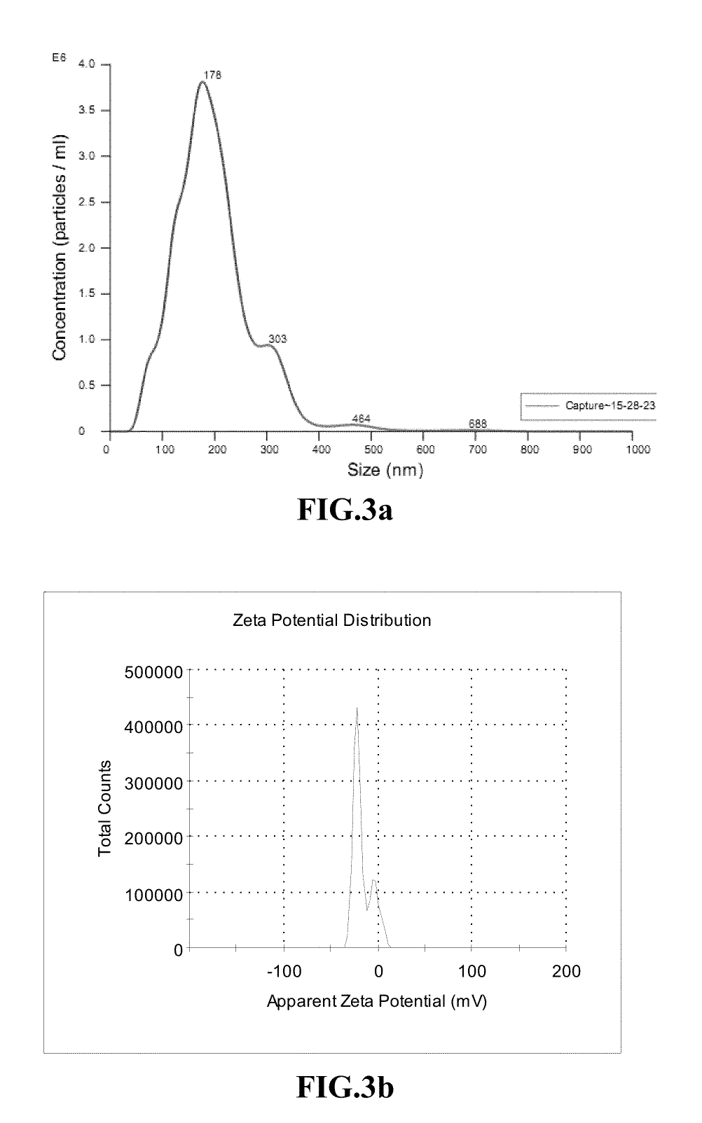

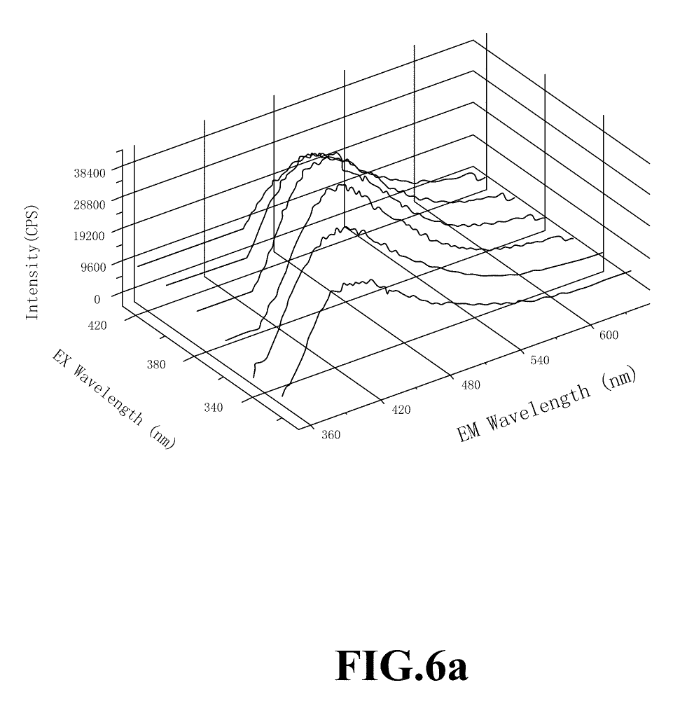

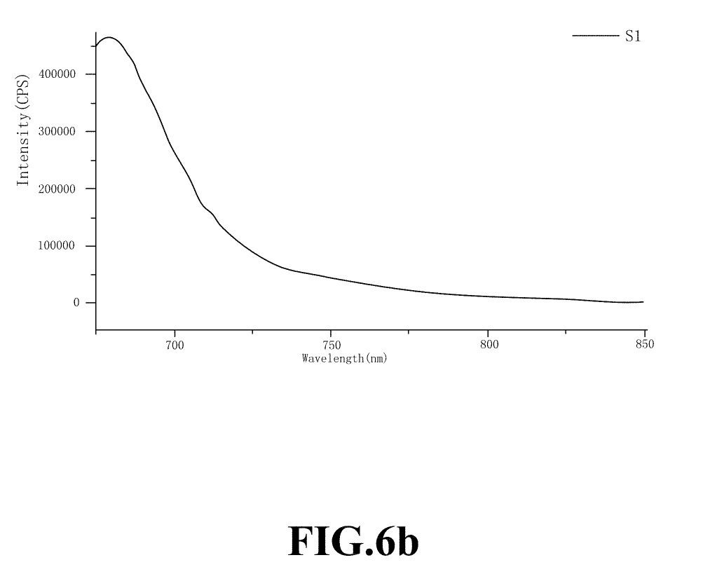

Embodiment 2: 10 milliliters of freshly brewed soy milk and 1 milliliter of methylene blue solution (0.5 milliliter/milliliters) are mixed, and then diluted 10 folds using ultra-pure water to obtain a precursor solution. The precursor solution is then placed in a 5 milliliter special glass bottle for use in microwave reaction instruments, and afterwards, the bottle is placed in a microwave reaction instrument, and the reaction conditions are set to 100 degrees centigrade and 5 hours. The reaction system is allowed to wait for 60 minutes, and then centrifugated, and the supernatant liquid is retained, and composite carbon nanodots are obtained following ultrafiltration. The methylene blue concentration measured in composite carbon nanodots using ultraviolet spectrophotometer standard curve method is 8.6 micrograms/milliliter. Measurements are made by a laser particle analyzer according to dynamic light scattering principle, and the grain size distribution of the composite carbon nanodots loaded on methylene blue are found as 197.9±79.7 nanometers (See, FIG. 3a), while the surface zeta potential is found as 31 21.5±4.36 millivolts (See, FIG. 3b). The fluorescence properties of composite carbon nanodots measured by a fluorescence detector device are shown in FIGS. 6a and 6b.

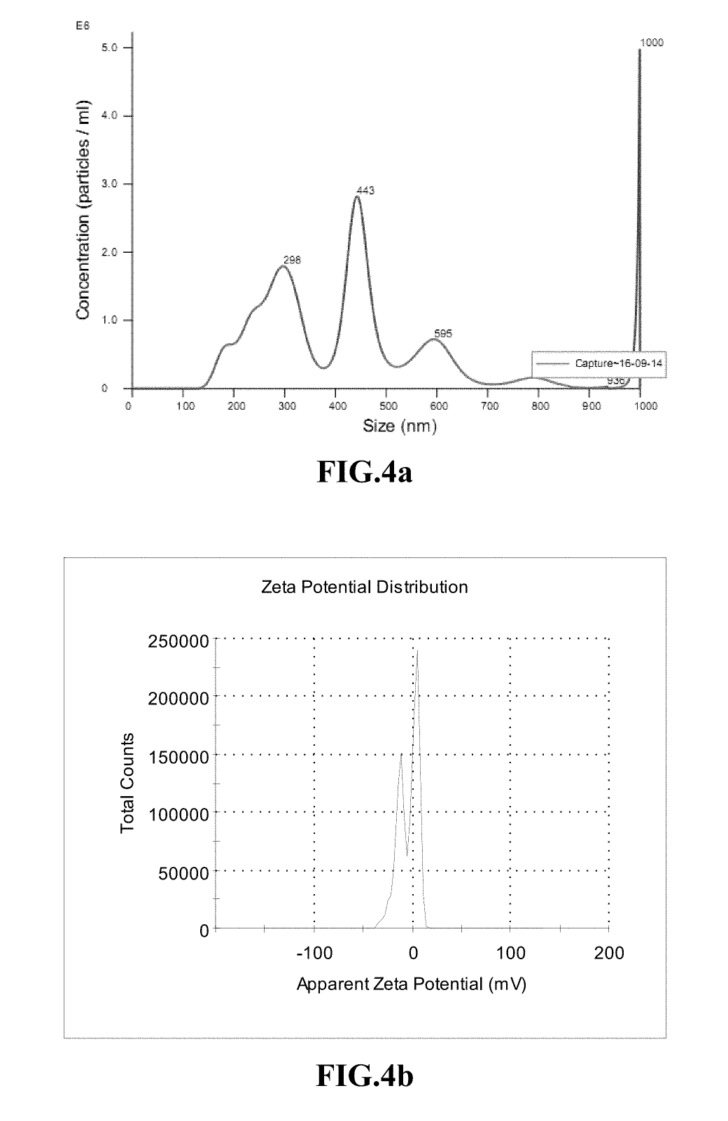

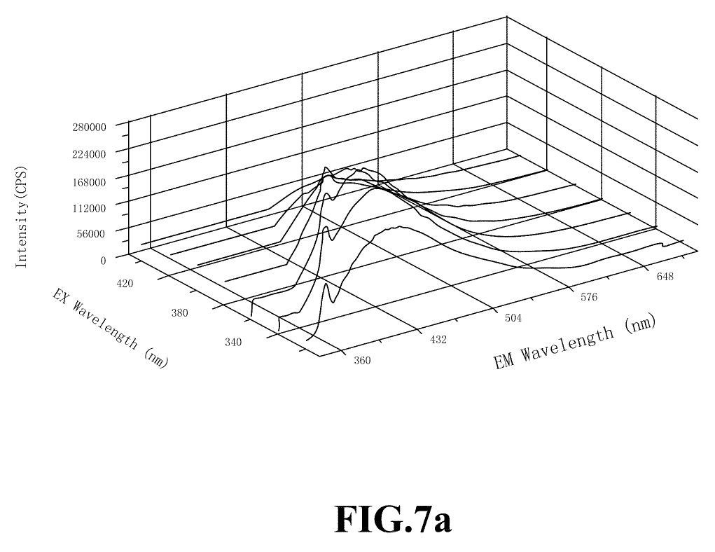

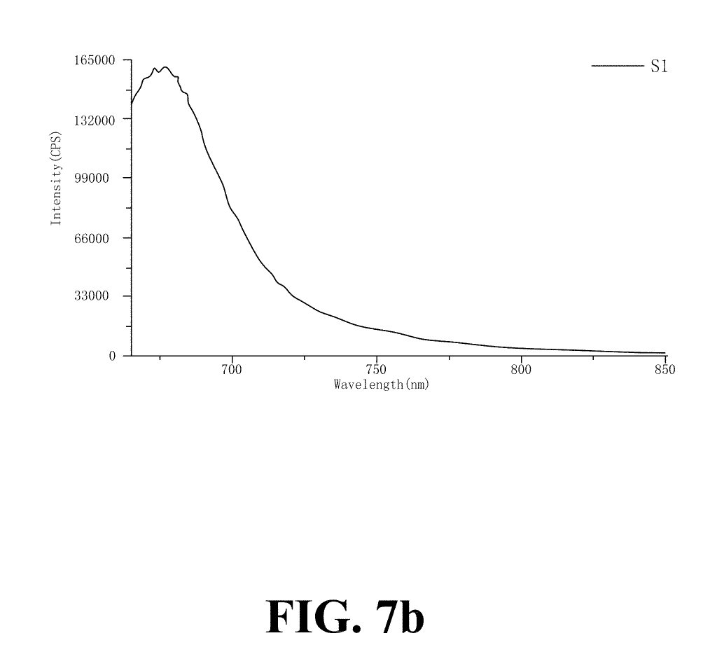

Embodiment 3: 1 milliliter of dietary milk and 10 milliliters of methylene blue solution (0.5 milligrams/milliliters) are mixed, and then diluted 5 folds using ultra-pure water to obtain a precursor solution. The precursor solution is then placed in a 5 milliliter special glass bottle for use in microwave reaction instruments, and afterwards, the bottle is placed in a microwave reaction instrument, and the reaction conditions are set to 160 degrees centigrade and 2 hours. The reaction system is allowed to wait for 1 hour, and then centrifugated, and the supernatant liquid is retained, and composite carbon nanodots are obtained following dialysis. The methylene blue concentration measured in composite carbon nanodots using standard curve method is 1.8 micrograms/milliliter. Measurements are made by a laser particle analyzer according to dynamic light scattering principle, and the grain size distribution of the composite carbon nanodots loaded on methylene blue are found as 434.4±196.8 nanometers (See, FIG. 4a), while the surface zeta potential is found as 4.92±2.88 millivolts (FIG. 4b). The fluorescence properties of composite carbon nanodots measured by a fluorescence detector device are shown in FIGS. 7a and 7b.

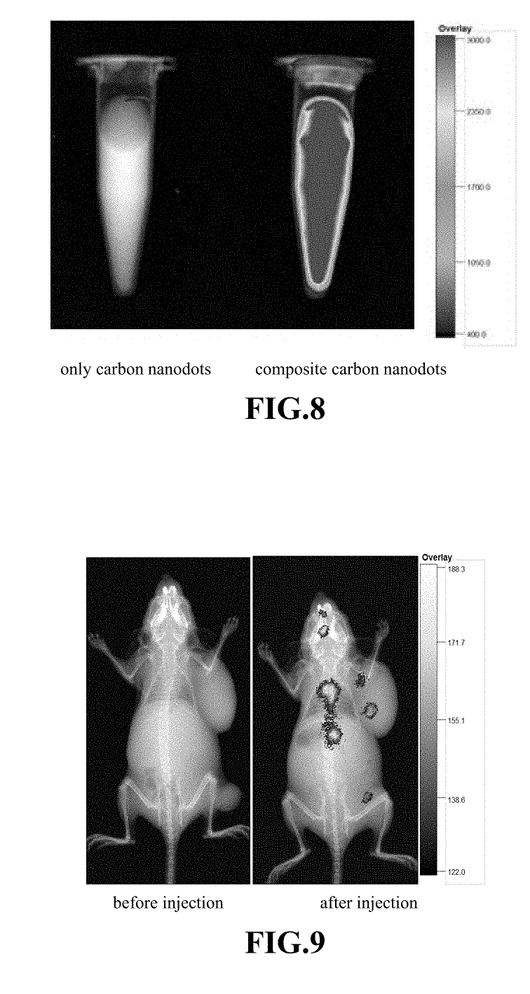

The present invention provides composite nanodots that are loaded on methylene blue and their use in the field of fluorescent imaging The combination of the composite nanodots based on carbon nanodots and the fluorescent dyes form composite fluorescent carbon nanodots, and the obtained composite nanometer fluorescence imaging materials have favourable biocompatibility and safety characteristics, provide high fluorescent imaging sensitivity, and in the fluorescent field, after being stimulated by near infrared light, they produce a fluorescent signal response as shown in FIG. 8. As indicated by in vivo imaging experiments on rats in near infrared light field, compared to blank groups (not loaded with carbon nanodots), the composite carbon nanodots loaded on methylene blue can remarkably increase the contrast and resolution of in vivo fluorescent imaging in animals (See, FIG. 9). They are promising in gaining wider use in the fields of biomedical imaging, targeting diagnosis and therapy, drug screening and optimization, and in vivo labelling and tracing, and also, they have potential value in the field of personalized medicine, and therefore, they are expected to have wide usage prospects in the field of biomedical imaging.

1. Composite nanodots based on carbon nanodots, characterized in that; the main components of the composition are carbon nanodots, and methylene blue is used as a support ligand.

2. Composite nanodots based on carbon nanodots according to claim 1, characterized in that; the carbon sources of the carbon nanodots in the composite nanodots are any of wolfberry leaching agents, soy milk, or dietary milk.

3. Composite nanodots based on carbon nanodots according to claim 1, characterized in that; the grain size range of the composite nanodots is 100-500 nanometers, and the zeta potential range is −30 to 10 millivolts.

4. Composite nanodots based on carbon nanodots according to claim 1, characterized in that; the concentration of methylene blue in composite carbon nanodots is 10 micrograms/milliliter.

5. A preparation method of composite nanodots based on carbon nanodots according to claim 1, characterized in that; it comprises the operation steps of:
a. forming a solution of the above said carbon sources and 0.5 milligrams/millilitres of methylene blue by means of mixing in the ratio of 0.1:1 to 10:1 by volume, diluting the mixture 1-10 folds with ultra-pure water, and thus obtaining a precursor solution;
b. placing the above said precursor solution in a microwave reaction instrument, and setting the instrument parameters as follows: temperature: 100-180° C., time: 30-300 minutes;
c. reacting for more than 30 minutes, obtaining a clear solution as a result of centrifugal separation, and obtaining crude product of composite carbon nanodots;
d. applying purification on the crude product of composite nanodots.

6. Method of preparation of composite nanodots based on carbon nanodots according to claim 5, characterized in that; purification is applied on the crude product of composite nanodots using ultrafiltration purification.

7. Method of preparation of composite nanodots based on carbon nanodots according to claim 5, characterized in that; purification is applied on the crude product of composite nanodots using dialysis purification.

8. Use of the composite nanodots based on carbon nanodots according to claim 1 in the field of fluorescence biological imaging.
